(19) United States
(12) Patent Application Publication
(10) Pub. No.: US 2006/0064022 A1
(43) Pub. Date: Mar. 23, 2006

(54) Tunnel type electronic sphygmomanometer

(75) Inventors: Paul Yang, Chung HoCity (TW); Shan-Yi Yu, Chung Ho City (TW)

Correspondence Address: BACON & THOMAS, PLLC, 625 SLATERS LANE, FOURTH FLOOR, ALEXANDRIA, VA, 22314, US

(73) Assignee: HEALTH & LIFE CO., LTD, CHUNG HO CITY (TW)

(21) Appl. No.: 10/944,772

(22) Filed: Sep. 21, 2004

(51) Int. Cl. A61B 5/02

(52) U.S. Cl. 600/490

(57) ABSTRACT

The present invention discloses a tunnel type electronic sphygmomanometer which comprises a base; an electronic manometer casing disposed on the base; a display device installed on the electronic manometer casing; and a blood pressure measuring device installed on one side of the electronic manometer casing; wherein the blood pressure measuring device is coupled to the electronic manometer casing through an electric circuit and a gas pipeline, such that the blood pressure measuring device is coupled by a fixture during the manufacturing process of the electronic sphygmomanometers, and sphygmomanometer manufacturers can meet the requirements for a blood pressure measurement taken from the left arm or the right arm of a user, wherein the electronic sphygmomanometer can be placed on either sides of the arm of the user as to achieve the objectives of effectively lowering the manufacturing cost and reducing the storage space for inventory.

### BACKGROUND OF THE INVENTION

1. Field of the Invention

The present invention relates to a tunnel type electronic sphygmomanometer, which allows users to change the position of the sphygmomanometer by means of its being fixed by a fixture, so that sphygmomanometer manufacturers do not need to produce different models of sphygmomanometers to meet the requirements for a blood pressure measurement taken from the left arm or the right arm of a user, and thus the invention can effectively lower the manufacturing cost and reduce the storage space for inventory.

2. Description of the Related Art

In general, the structure of a traditional tunnel type electronic sphygmomanometer comprises a casing; a measuring fixed base being disposed on one side of the casing and integrally coupled with the measuring fixed base and having a tunnel that passes through the measuring fixed base, such that a user can pass one of their arms through the tunnel; an annular air bag being disposed around the internal periphery of the tunnel; a sensor being installed in the air bag for detecting the pressure signal during a blood pressure measurement; a display device, an electric device and an air pump being installed on the other side of the measuring fixed base and coupled with the air bag and the sensor in the casing through an electric circuit and a gas pipeline, and the display device displays the result of the blood pressure measurement. Therefore, different models for the measurements taken from the left arm or the right arm of a user will be taken into consideration for the manufacture of the tunnel type electronic sphygmomanometer. Since the measuring fixed base is integrally coupled with the casing, therefore when the traditional tunnel type electronic sphygmomanometer is manufactured, it is necessary to build different molds for different models to meet customer's requirements, which will increase the manufacturing cost and the storage space of inventory.

### SUMMARY OF THE INVENTION

In view of the foregoing shortcomings of the prior art, the inventor of the present invention focused on the problem to start finding a way for the improvement and overcome the shortcomings in hope of finding a feasible solution, and conducted extensive researches and experiments and finally invented the tunnel type electronic sphygmomanometer in accordance with the present invention that allows users to change the left and right positions of the measuring device.

Therefore, it is the primary objective of the present invention to provide a tunnel type electronic sphygmomanometer, which comprises a base; an electronic manometer casing disposed on the base; a display device installed on the electronic manometer casing; and a blood pressure measuring device installed on one side of the electronic manometer casing; wherein the blood pressure measuring device is coupled to the electronic manometer casing through an electric circuit and a gas pipeline and the blood pressure measuring device is coupled with the electronic manometer casing by a fixture such that the blood pressure measuring device can be placed on either the left side or the right side of the electronic sphygmomanometer as to comply with the ergonomic engineering requirements and the differential blood pressure measured from the left or right arm of a user. Thus, the invention can work with different models of sphygmomanometers and achieve the objectives of effectively lowering the manufacturing cost and reducing the storage space for inventory.

### BRIEF DESCRIPTION OF THE DRAWINGS

The above objects, features and advantages of the present invention will become apparent from the following detailed description taken with the accompanying drawing.

FIG. 1 is a perspective view of the tunnel type electronic sphygmomanometer according to the present invention.

FIG. 2 is a perspective view of the tunnel type electronic sphygmomanometer according to a preferred embodiment of the present invention

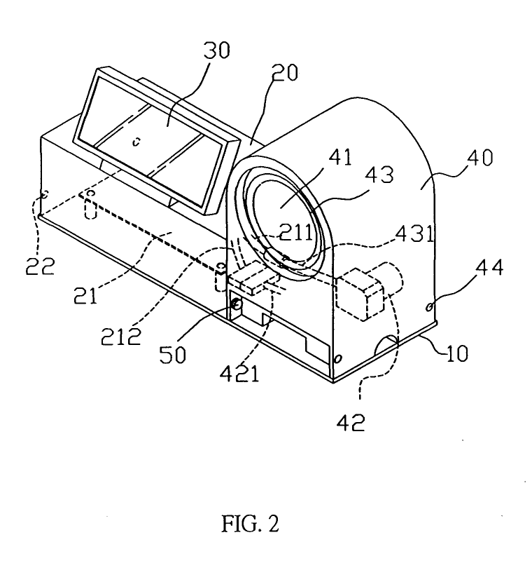

FIG. 3 is another perspective view of the tunnel type electronic sphygmomanometer according to a preferred embodiment of the present invention.

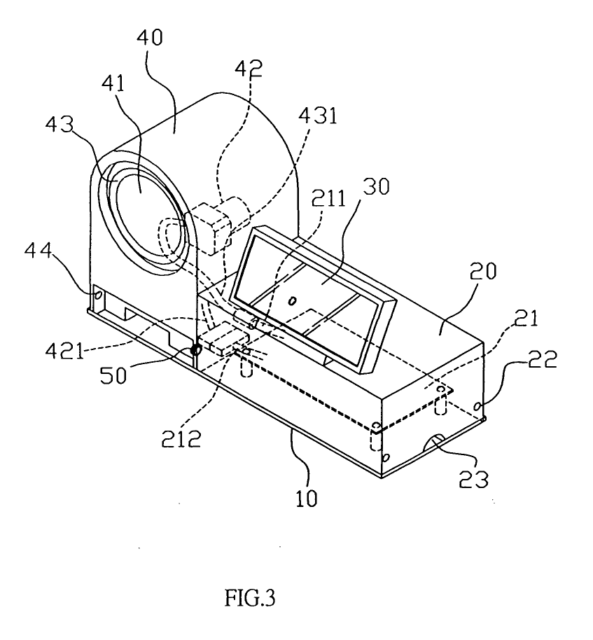

### DETAILED DESCRIPTION OF THE PREFERRED EMBODIMENTS

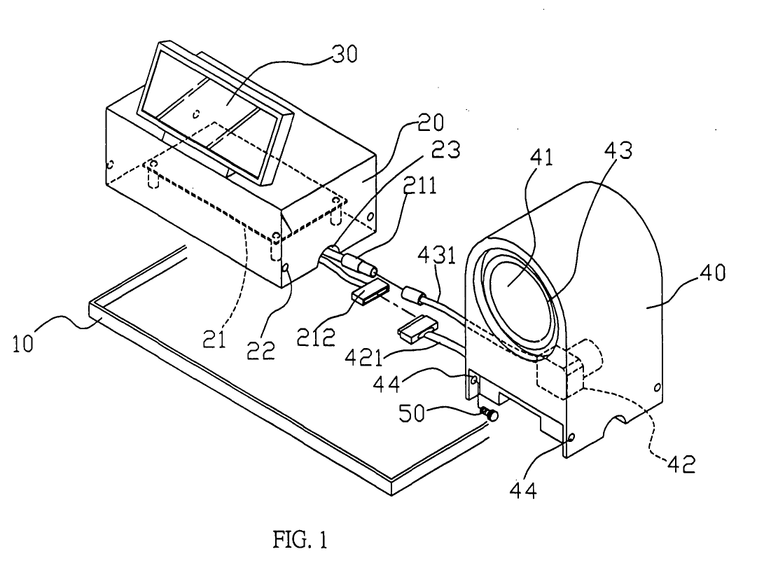

Please refer to FIG. 1 for a tunnel type electronic sphygmomanometer according to the present invention. The tunnel type electronic sphygmomanometer comprises: a base 10; an electronic manometer casing 20 being disposed on the base 10; a circuit board 21 being installed in the electronic manometer casing 20; a gas connector 211 and an electric connector 212 being disposed on the circuit board 21, and the gas connector 211 and the electric connector 212 extending outward from a groove 23 on one side of the electronic manometer casing 20; a display device 30 being installed on the electronic manometer casing 2 and electrically connected to the circuit board 21 by an electric circuit; and a fixture 22 disposed individually on both sides of the electronic manometer casing 20, which is a screw adopted by this embodiment and the persons skilled in the art may substitute it by other fixing device such as a hook and the like.

Further, the base 10 has a blood pressure measuring device 40 installed on one side of the electronic manometer casing 20, and the blood pressure measuring device 40 has a hollow tunnel 41 and a motor device 42 therein; and the tunnel 41 allows the arm of a user to pass through, and the tunnel 41 has an annular air bag 43 disposed around the internal periphery of the tunnel 41. An air connector 431 is disposed on the air bag 43 and the air connector 431 is connected to a gas connector 211 in the electronic manometer casing 20, and the air bag 43 is driven by the motor device 42, and the air bag 43 is expanded or contracted by the rotation of the motor device 42. An electric connector 421 is disposed on the motor device 42, and the electric connector 421 is connected precisely to the electric connector 212 in the electronic manometer casing 20. The circuit of the electronic manometer casing 2 controls the rotation of the motor device 42. Further, a fixture 44 is disposed individually on both sides of the blood pressure measuring device 40, which is a screw adopted by this embodiment and the persons skilled in the art may substitute it by other fixing device such as a hook and the like.

Therefore, the blood pressure measuring device 40 is coupled with the electronic manometer casing by a fixture 50 (which is a screw in this embodiment) and a fixing device 22, 44 such that the blood pressure measuring device 40 can be placed on either the left side or the right side of the electronic manometer casing 20 as to comply with the ergonomic engineering requirements and the differential blood pressure measured from the left or right arm of a user. Thus, the invention can work with different models of sphygmomanometers and achieve the objectives of effectively lowering the manufacturing cost and reducing the storage space for inventory.

In summation of the above description, the present invention herein enhances the performance than the conventional structure and further complies with the patent application requirements and is submitted to the Patent and Trademark Office for review and granting of the commensurate patent rights.

While the invention has been described by way of examples and in terms of preferred embodiments, it is to be understood that the invention is not limited thereto. To the contrary, it is intended to cover various modifications and similar arrangements and procedures, and the scope of the appended claims therefore should be accorded the broadest interpretation so as to encompass all such modifications and similar arrangements and procedures.

What is claimed is:

1. A tunnel type electronic sphygmomanometer, comprising: a base;
an electronic manometer casing; being disposed on one side of said base and having a display device;
a blood pressure measuring device; being disposed on the other side of said base and coupled with said electronic manometer casing through an electric circuit and a gas pipeline; and
a fixture, for fixing said electronic manometer casing with said blood pressure measuring device;
thereby when said tunnel type electronic sphygmomanometer is in use, the position of said blood pressure measuring device is changed by means of the fixing of said fixture to fit its disposition space.

2. The tunnel type electronic sphygmomanometer of claim 1, wherein said electronic manometer casing and said blood pressure measuring device have a fixing device corresponding to said fixture being individually disposed on both sides.

3. The tunnel type electronic sphygmomanometer of claim 2, wherein said fixing device is a screw hole.

4. The tunnel type electronic sphygmomanometer of claim 1, wherein said fixture is a screw rod.
